(19) United States
(12) Patent Application Publication
(10) Pub. No.: US 2012/0276070 A1
(43) Pub. Date: Nov. 1, 2012

(54) Induced Pluripotent Stem Cells and Related Methods

(75) Inventor: James Musick, Conifer, CO (US)

(73) Assignee: VITRO DIAGNOSITICS, INC., Golden, CO (US)

(21) Appl. No.: 13/510,486

(22) PCT Filed: Nov. 17, 2010
(86) PCT No.: PCT/US10/56986

§ 371 Date: Jun. 25, 2012

Related U.S. Application Data
(60) Provisional application No. 61/261,967, filed on Nov. 17, 2009.
(60) Provisional application No. 61/304,875, filed on Feb. 16, 2010.

(51) Int. Cl. C12N 5/071; C12N 5/0775; C12N 5/0789; C12Q 1/02; A61K 35/12; A61P 1/00; A61P 35/02; A61P 3/10; A61P 25/28; A61P 25/16; A61P 19/02; C12N 5/078; A61P 35/00

(52) U.S. Cl. 424/93.7; 435/377; 435/29

(57) ABSTRACT

The present invention provides materials and methods to reprogram adult stem cells without transfection of foreign genes through the employment of appropriate environmental factors. Cells made via these processes are also provided. Methods to use the pluripotent cells are also provided.

### CROSS-REFERENCE TO RELATED APPLICATIONS

This application claims the benefit of U.S. Provisional Application No. 61/261,967, filed Nov. 17, 2009, and U.S. Provisional Application No. 61/304,875, filed Feb. 10, 2010 the disclosures of which are incorporated herein by reference.

### STATEMENT OF FEDERAL SPONSORSHIP

No federal sponsorship of the research is related to this invention.

### FIELD OF THE INVENTION

The present invention is in the field of stem cells, particularly stem cell generation and maintenance. Therefore, the field includes cell physiology, culture medium technology, and molecular biology.

### BACKGROUND OF THE INVENTION

Stem cells are characterized by two primary properties: self-renewal and an ability to differentiate into various different cell types such as blood, muscle, nerve cells, etc. Self-renewal refers to proliferation or cell growth of stem cells without ensuing differentiation. Self-renewal may be limited to a set number of generations as in adult stem cells or extend to perpetuity, called immortality, in embryonic stem cells.

Embryonic stem cells exhibit pluripotent differentiation capacity that is defined as the ability to differentiate into any cell of the body while adult stem cells exhibit limited differentiation capacity called multipotent differentiation, referring to limited differentiation capacity to form only specific cell types, as illustrated, by hematopoietic or mesenchymal stem cells that form various blood cell types or primarily bone, cartilage and fat cells, respectively.

Through extensive research into the genetic basis of “stemness” oriented toward determining the genes responsible for stem cell properties of self-renewal and the ability of these cells to differentiate into different cell types, procedures were discovered that allowed reprogramming of adult cells into cells with properties of embryonic stem cells. These cells are termed “induced pluripotent stem cells” or iPS or iPSC. Induced pluripotent stem cells (iPSC) enable pluripotentiality through the reprogramming of adult cells thus avoiding use of embryonic cells and the associated ethical dilemmas.

IPSCs exhibit properties of embryonic stem cells including continuous self-renewal and an ability to differentiate into any type of cell in the body (pluripotentiality). The original methods of iPSC generation involved transfection of human skin fibroblasts. IPSC technology has considerable clinical potential in the generation of personalized, autologous cells with pluripotent differentiation capacity and thus numerous medical applications. However, clinical application of iPSC technology requires methods of iPSC generation without transfection of target cells with foreign genes to ensure safety.

Here, the inventors describe simplified methods for the generation of iPSC without transfection of foreign genes.

### SUMMARY OF THE INVENTION

The present invention provides materials and methods to reprogram adult stem cells without transfection of foreign genes through the employment of appropriate environmental factors. Cells made via these processes are also provided.

The present invention provides methods comprising culturing adult stem cells at less than 20% v/v oxygen so as to induce pluripotency, particularly provided are those methods wherein the adult stem cells are cultured at from about 1% v/v to about 5% v/v oxygen, particularly provided are those methods wherein the adult stem cells are cultured at from about 1% v/v to about 3% v/v oxygen, particularly provided are those methods wherein the adult stem cells are cultured at from about 1% v/v to about 2% v/v oxygen, most particularly provided are those methods wherein the adult stem cells are cultured at approximately 1% v/v oxygen.

Also provided are those methods wherein the balance of the gas phase is nitrogen.

Also provided are those methods which further comprise culturing in the presence of a small molecule that promotes expression of POU5-F1 (Oct3/4), particularly those wherein the small molecule is selected from the group consisting of: valproic acid; 5-azacytidine; sodium butyrate; gonadotropin releasing hormone; ascorbic acid (Vitamin C); antisense or sense miRs of the miR-290-295 cluster; and hydrocortisone.

Also provided are those methods wherein the adult stem cells are selected from the group consisting of: hematopoietic stem cells; mesenchymal stem cells of any origin; bone; adipose; umbilical cord; or peripheral blood; Warton's jelly of the umbilicus; skin; decidua of the placenta; amniotic fluid; teeth (both juvenile and adult); vasculature; muscle (including myogenic satellite cells); and endocrine gland, particularly those wherein the adult stem cell is a mesenchymal stem cell.

Also provided are those methods which further comprise a step of identifying pluripotency attributes of the cultured cells.

Also provided are those methods which further comprise a step of purifying pluripotent cells.

Also provided are those methods which further comprise a step of identifying POU5-F1 (Oct3/4) expression.

Also provided are pluripotent cells made by the methods herein.

Also provided are methods useful to identify therapeutic compounds, comprising culturing a cell according to the method of claim 1, introducing a test therapeutic compound, and determining if the test therapeutic compound is useful.

Also provided are methods useful to treat a disease, comprising administering a pluripotent or differentiated cell herein, particularly wherein the disease is selected from the group consisting of: leukemia; lymphoma; diabetes; Alzheimer's; Parkinson's; multiple sclerosis; osteoarthritis; stroke; myocardial infarction; congestive heart failure; graft-verses-host disease; traumatic brain injury; Crohn's disease; stem cell-mediated malignancy; age-related hearing loss; macular degeneration; spinal cord injury; end-stage renal disease; acute or chronic renal failure; diabetes-related cardiovascular disease; ALS; spinal cord injury; herniated disc; ligament or tendon rupture.

Also provided are methods comprising differentiating a pluripotent cell herein, particularly those wherein the differentiated cell is selected from the group consisting of: cardiomyoctes; various neuronal cells including neurotransmitter-specific neurons; sensory neurons; alpha motor neurons; Schwann and glial cells; hepatocytes; vascular endothelial cells; skeletal and smooth muscle cells; renal tubule cells; glomerular cells of the nephron; kerotinocytes; osteoblasts; adipocytes; chondrocytes; pituitary hormone producing cells; thyroid and adrenal cells; melanocytes; thymocytes; erythrocytes, neutrophils, basophils, eosinophils, macrophages, platelets and T- and B-lymphocytes. epithelial cellular systems of the gut and urogenital tract.

Differentiated cells made according to the present invention are also provided.

Various aspects of this invention will become apparent to those skilled in the art from the following detailed description of the preferred embodiment, when read in light of the various examples and accompanying drawings contained herein.

### BRIEF DESCRIPTION OF THE DRAWINGS

FIG. 1: Effect of oxygen level on MSC growth: Compare 5% and 20% O₂ with Cross-over Protocol. The doubling time of human MSCs is shown as a function of passage number. Passage 5, 6 & 7 MSCs were cultured as described and sub-cultured when the cultures at 5% O₂ reached 90-95% confluence. Passage 5 included changing cells from 5% to 20% O₂ and those in 20% to 5% O₂ together with direct transfer to 5% or 20% O₂. Doubling time is shown through three successive passages.

FIG. 2: Selection of transfectants. This figure shows human MSCs prior to transfection with POU5F1 containing Lentiviral expression vector (Pre-transfection) and 3 days following selection in puromycin (day 4) when non-transfectants were killed. Day 8 represents 7 days in selection medium and Day 10 represents 9 days in selection medium. Note possible adipocyte-like structures at Day 10.

FIG. 3: Growth rate of POU5-F1 transfectant and native MSC. Clone H12 cells and native human MSCs were maintained in culture and doubling times were determined as described.

FIG. 4: Differentiation of POU5-F1 Transfectants (lower panels) and native MSCs (upper panels) into chondrocytes (left panel) adipocytes (center panel) and osteoblasts (right panel). MSCs and transfectants were cultured and then stained for lineage-specific cell types as described. The results show that both native and transfected MSCs exhibit multipotent differentiation capacity.

FIG. 5: Stem cell potency assay: LumiSTEM™-96. This shows potency determination of clone A7, (squares) and native human MSCs (triangles and circles) or human fibroblasts (inverted triangles) as described. The results show a significant increase in the slope of the dose-response curve of transfectants suggesting an increase in differentiation capacity.

### DETAILED DESCRIPTION OF THE INVENTION

The present invention is based in part on the discovery that transfection of an adult stem cell, specifically, a human mesenchymal stem cells (MSC), with an expression vector that enhanced the expression of POU5-F1 (also known as Oct3/4) is able to become pluripotent if cultured under particular conditions. The effects of POU5-F1 in MSCs were previously unknown. Surprisingly, over-expression of POU5-F1 altered the MSCs to become pluripotent. The transfected MSCs not only maintained multipotent differentiation capacity but also exhibited expanded differentiation capacity that is characteristic of iPSCs. The discovery demonstrates that over-expression of Oct3/4 reprogrammed adult stem cells to pluripotency. This is in contrast to somatic cells, which require over-expression of four different pluripotentiality genes (ectopic expression of Oct3/4, Sox2, Kp14 & c-myc) in order to become reprogrammed.

Methods to Generate Induced Pluripotent Stem Cells (IPSCs).

The present invention includes use of adult stem cell cultures as the cellular source for generation of iPSCs. Preferred methods involve stimulation of endogenous POU5-F1 (Oct3/4) expression without transfection of foreign DNA. The present invention teaches methods of iPSC generation through the culture of adult stem cells in environments that are particularly suited to induce pluripotency through appropriate physical aspects of the environment especially its gas phase, appropriate media for maintenance of the adult stem cell culture together with the addition of specific small molecules to the media which result in regulation of the expression of endogenous pluripotency genes to ensure pluripotent developmental capacity. Thus, for example, appropriate environmental conditions are employed to induce endogenous expression of POU5-F1 while maintaining sufficient expression of additional pluripotency genes to enable reprogramming to the pluripotent state.

The present methods include a step of maintaining adult stem cell cultures at about 1% O₂ through use of a gas phase for cell culture comprised of 1% O₂, 5% CO₂ and 94% N₂. The O₂ content may vary from 1 to 1.5% while the CO₂ content not critical and is used to maintain physiological pH of the medium when NaHCO₃ is used as a buffer. The balance of the gas phase is preferably N₂.

The present invention may be performed in a maintained gas phase wherein the O₂ content is less that 20% (v/v), more preferably between 1% to 5% O₂ (v/v) and most preferably at approximately 1% O₂ (v/v). Minor variations from these stated levels are within the scope of the present invention. However, it will be recognized by those skilled in the art that deleterious hypoxic conditions are to be avoided, <1% O₂ since such conditions are toxic and inhibit cell growth.

In one embodiment, the method includes maintaining cultures of adult stem cells as described in a state of continual shelf renewal which is manifested by cellular growth rates that are characteristic of the stem cells. Thus, for example human MSCs derived from cord blood, adipose tissue or teeth exhibit doubling times from 20 to 30 hours which is consistently maintained for at least 4 to 5 continuous passages.

Small Molecule Enhancers.

A further embodiment includes culture in a reduced O₂ environment (1 to 1.5%) together with agents added to the medium targeting positive regulation of the Oct3/4 promoter. The Oct4 (POU5-F1 & Oct3 also) promoter is known to consist of four conserved regions, CR1, 2, 3 & 4 consisting of both positive and negative regulatory elements. A retinoic acid binding region is thought to mediate suppression of Oct4 expression, as occurs during differentiation, while positive response elements include binding regions to Sp1, Sp3 transcription factors and a hormone response element that is positively regulated through the steroidogenic factor-1 transcription factor. The endogenous level of Oct4 expression is determined through a complex interplay of various transcription factors, epigenetic factors and chromatin influences. The present invention includes use of small molecular agents added to the culture medium that result in positive regulation of Oct4 expression and hence in reprogramming to the pluripotent state.

A preferred embodiment includes the inclusion of the valproic acid at concentrations ranging from 0.2 to 5 mM, most preferably at 1 to 2 mM. VPA is known to positively regulate Oct 4 expression probably through binding to the hormone response element of its promoter and this effect appears independent of its inhibition of histone deacetylases resulting in modulation of chromatin. The present invention may optionally include use of GnRH to increase Oct 4 expression through actions on its promoter. The preferred concentration of GnRH is 75 pg/ml with a preferred concentration range of 50 pg/ml to100 pg/ml. There is evidence that GnRH enhances expression of SF-1, a positive regulatory factor of the Oct 4 promoter.

Also, the present invention optionally includes use of 5-azacytidine to demethylate DNA and thus relieve DNA methylation barriers to reprogramming as a small molecular agent to induce pluripotency in adult stem cells. Preferably 5-aza-cytidine is added to the medium at 0.1 to 0.6 mM, most preferably at 0.4 to 0.5 mM. Administration of 5-azacytidine is optimally transient in the reprogramming process, preferably lasting 24 to 48 hours especially at later stages of the pluripotent reprogramming process following the formation of pre-iPS structures as indicated for example by the presence of SSEA1-positive cells.

Likewise, sodium butyrate may relieve chromatin barriers to reprogramming through inhibition of histone deacetylase and may optionally be present at 1 to 5 mM in the medium and most preferably at 1 to 2 mM.

Another optional small molecule media addition encompassed herein is ascorbic acid (Vitamin C), preferably at 25 to 100 μM and most preferably at 50 to 60 μM.

Furthermore, embodiments include optional addition of hydrocortisone to the adult stem cell culture medium preferably at 80 nM to 110 nM but including a range of concentrations from 30 nM to 300 nM.

Also, induction of adult stem cells through introduction of the microRNA family known as the miR-290-295 cluster is within the scope of this invention.

Use of several, all, none, or combinations of the above small molecules are within the scope of the present invention.

Adult Stem Cells Useful in the Present Invention.

The present invention includes the use of adult stem cells, which in the broadest sense, is intended to include any adult stem cell obtained from any animal species. Adult stem cells as used herein, refers to any non-embryonic stem cell possessing the properties of self-renewal and multipotent differentiation capacity. Thus, an adult stem cell is any non-embryonic progenitor cell capable of proliferation without differentiation and subsequent differentiation into specific terminally differentiated cell types under the influence of appropriate conditions.

Such adult stem cells include, but are not limited to: a) hematopoietic stem cells capable of differentiation into blood cell types including: erythrocytes, neutrophils, basophils, eosinophils, macrophages, platelets and T- and B-lymphocytes. b) Mesenchymal stem cells present in bone marrow, adipose tissue, umbilical cord or peripheral blood, Warton's jelly of the umbilicus and also within various other tissues such as skin, decidua of the placenta, amniotic fluid, teeth (both juvenile and adult), vasculature, muscle cells as satellite cells and endocrine glands. MSCs are known to differentiate into chondrocytes, adipocytes and osteoblasts as well as stromal cells, muscle and nerve cells. c) neural stem cells as present within the subventricular zone, subgranular zone of the hippocampal dendate gyms, olfactory epithelium, and peripheral neural structures such as the carotid body, d) satellite cells of muscle including, skeletal, cardiac and smooth muscle e) endocrine-specific adult stem cells including U.S. Pat. No. 7,527,977 f) endothelial adult stem cells.

The species of adult stem cells within the present invention include, but are not limited to following mammalian species: human, mouse, rat, canine, feline, equine, bovine, porcine. Also included are adult stem cells derived for other species including birds, fishes, and reptiles.

Extraction and Purification of Stem Cells.

The procedures for extraction and purification of adult stem cells are known in the art. Thus for example, adult stem cells may be isolated by aspiration from specific compartments such as bone marrow, extracted from various tissues containing adult stem cells by a combination of surgical excision, mechanical and enzymatic dissociation of tissues into cellular dispersions contained within various fluids commonly used for preparation of such cellular dispersions, including but not limited to, phosphate buffered saline containing appropriate concentrations of collagenase. Other procedures for achieving cellular dispersions are readily apparent to those skilled in the art and the present invention is not limited to particular methods of obtaining a cellular dispersion from a source of adult stem cells.

Stem cell purification from a cellular mixture is also may be accomplished by any procedure. Separation methods based on physical cellular properties such as density gradient centrifugation, differential adsorption, limited dilution cloning and related procedures may be used to purify adult stem cells within the context of the present invention. More selective procedures including those based on the expression of specific molecules on the surface of adult stem cells may be employed in the purification.

Adult stem cells may be characterized by specific phenotypic properties including expression of cluster designation antigens expressed on the cell surface and such expression forms the basis of purification methods that may be based on positive or negative selection. For example, antibodies to specific antigens may be coupled to magnetic particles allowing purification of cells expressing specific cluster designation antigens. Flow cytometry methods may also be useful in the purification of adult stem cell populations by their enrichment relative to other cells.

Purified adult stem cells may be maintained in cell culture by methods well-known to the practitioners of cell culture. Cells may be plated onto a surface substrate including specialty plastics, extracellular matrix material in the case of adherent cells and bathed in cell culture medium, a specialty fluid designed to support the nutritional, energetic and growth needs of the culture. Suspension cultures may likewise be maintained but with the cells suspended within the cell culture medium. The culture plate, dish or flask may then be placed within an incubator to maintain temperature, relative humidity and gas phase. The cultures may optionally be microscopically monitored periodically to determine cellular morphology, growth, degree of confluence, etc. Variations in plating density are also well-known to those skilled in the art and all variations of plating density, especially including low plating densities at less than 200 cells/cm², are encompassed within the present invention.

Maintaining Cultures of Adult Stem Cells.

In-vitro cultures of adult stem cells may be maintained by use of appropriate environmental conditions that are designed to mimic conditions of the in vivo environment from which the adult stem cells are derived. Several commercially available media provide support and maintenance of adult stem cells in culture.

However, it is preferable to use a medium that maintains adult stem cells in self-renewal without terminal differentiation. Medium choice is determined by the adult stem cell used as the starting material for the present invention, e.g., hematopoietic stem cells rapidly differentiate into specific blood cell lineages while MSCs may be maintained for extended passages in cell culture without lineage specific differentiation in the absence of specific agents that induce differentiation.

Cell culture media may contain serum or not. Serum-free media formulations are preferred for clinical applications of the present invention since the exposure to potential adventitious agents is eliminated through the use of chemically defined, animal-component free media.

A variety of attachment surfaces are suitable for maintaining adult stem cell cultures including both untreated and attachment factor-coated specialty plastic surfaces that are widely available for cell culture applications.

Analytical Methods to Determine and Validate the Generation of Authentic iPSCs.

Methods to detect early cellular changes that precede complete iPS reprogramming include increased growth rate, reduction in cell size, expression of alkaline phosphatase and SSEA-1 and colony formation that are characteristic of the pre-iPS phenotype. Appearance of such pre-iPS phenotypic characteristics may be used as guidelines for implementation of the methods taught through the present invention for iPS generation.

Dosage and length of exposure to the various small molecules used to induce the pluripotent state may be adjusted according to appearance of pre-iPS phenotypic characteristics. Variables, such as exact source of the adult stem cells, their age, the exact medium used for culture, etc may optionally be taken into account. Furthermore, various cultures of adult stem cells may optionally include singular or multiple simultaneous additions of small molecules to the medium at a limited period of time in the process.

The pluripotent state is well known to those skilled in the art and characterized by several properties including the ability to form three germ layers in both in-vitro and in-vivo environments, characteristic expression levels of pluripotency genes and demonstrated pluripotent differentiation capacity comparable to embryonic stem cells.

Methods to Use iPSCs.

Since the present invention provides methods to generate iPSCs from any cell type, the present invention also comprises a wide variety of methods to use the cells generated. For example, terminally differentiated cells may be created from the present invention, including cardiomyoctes; various neuronal cells including neurotransmitter-specific neurons; sensory neurons; alpha motor neurons; Schwann and glial cells; hepatocytes; vascular endothelial cells; skeletal and smooth muscle cells; renal tubule cells; glomerular cells of the nephron; kerotinocytes; osteoblasts; adipocytes; chondrocytes; pituitary hormone producing cells; thyroid and adrenal cells; melanocytes; thymocytes; erythrocytes, neutrophils, basophils, eosinophils, macrophages, platelets and T- and B-lymphocytes, epithelial cellular systems of the gut and urogenital tract. Such cells have considerable application to drug discovery and development by providing consistent cellular systems for toxicological studies, i.e., cell-based toxicity products, and for systems used in screening of various drug candidates, studies of drug interactions and mechanistic investigations of drug activity.

Moreover, iPSCs generated by the present methods have numerous therapeutic applications. For example, the methods taught by the present invention enable patient-specific generation of iPSCs that may be cryopreserved as a repository for future applications as needed by the patient. These include numerous therapeutic applications as will be apparent to those skilled in the art such as for example, leukemia; lymphoma; diabetes; Alzheimer's; Parkinson's; multiple sclerosis; osteoarthritis; stroke; myocardial infarction; congestive heart failure; graft-verses-host disease; traumatic brain injury; Crohn's disease; stem cell-mediated malignancy; age-related hearing loss; macular degeneration; spinal cord injury; end-stage renal disease; acute or chronic renal failure; diabetes-related cardiovascular disease; ALS; spinal cord injury; herniated disc; ligament or tendon rupture.

The results presented in the following examples describe cell culture parameters that increase the expression of POU5-F1 (Oct3/4) in adult stem cells, and also result in expanded differentiation capacity that is a characteristic of iPSCs. This was demonstrated by the transfection of a Lentiviral expression vector into human cord blood-derived MSCs which resulted in clonal cell lines that were expanded in medium containing 10 μg/ml puromycin that is lethal to wild type MSCs. The transfectants exhibited multipotent differentiation capacity to form chondrocytes, adipocytes and osteoblasts as did native, non-transfected MSCs. Also, by examination of stem cell potency through dose-response determination of cellular ATP, MSCs transfected with POU5-F1 showed a substantial increase in slope (p<0.0001, by analysis of covariance) indicating increased stem cell potency and hence differentiation capacity.

It will thus be apparent to those skilled in the art that endogenous activation of POU5-F1 is now a method for reprogramming of adult stem cells to pluripotency based on the demonstration of single factor (Oct4 itself) induction of the pluripotent state. The endogenous expression of POU5-F1 as shown in Example 1 was relatively low. However, the promoter region of the Oct4 gene is well-characterized and known to consist of 4 highly conserved regions that mediate positive or negative regulatory influences on POU5-F1 expression. Hence, the use of small molecules to increase POU5-F1 expression is an optional optimization embodiment of the present invention.

### EXAMPLES

### Example 1

### Endogenous Expression of Pluripotency Genes in Human Fibroblasts

The cell line known as VIT1 arose from a primary culture of dispersed human fetal pancreatic cells under conditions that select for growth of fibroblast cells. A single pancreas gland at 18 weeks gestation (Advanced Biosciences Resources; Alameda, Cailf.) was dispersed by micro-dissection and collagenase digestion (2 mg/ml for 50 minutes at 37° C.). Following washout of enzyme, cells were frozen down at about 1° C./minute in α-MEM, 10% FBS & 7.5% ethylene glycol and stored in liquid N₂. Frozen cells were subsequently rapidly thawed at 37° C., washed with 10 mls PBS and plated at a low density (˜200/cm²), favoring the selection of fibroblasts, in VitroPlus II growth medium (Vitro Diagnostics, Inc, Catalog Number VC03014).

Gene expression profiling of RNA derived from VIT1 cells in passage 4 was determined using the CodeLink microarray (Amersham). This analysis showed that 14,842 genes were expressed at levels that were greater than threshold out of a total of 20,000 human genes detectable with this array. Table 1 shows the level of expression of common pluripotency genes within this human fibroblast cell line.

| Gene | Expression Level * |
|---|---|
| POU5F1 (Oct3/4) | 1.74 |
| Klf4 (Gklf) | 26.2 |
| c-myc | 18.5 |
| Sox2 | ND |
| Sox1 | 11.98 |
| Sox3 | 0.91 |

* The threshold of detection was 0.29

These results show endogenous expression of pluripotency genes within cultured human fibroblasts. Both Klf-4 and c-myc were expressed at high levels while the level of POU5F1 was lower, but yet substantially above threshold detection levels. Sox 2 was not present on the microarray while both Sox-1 and Sox 3 were expressed.

These data thus showed measurable endogenous levels of key pluripotency genes and led to additional experiments aimed at determining the effects of ectopic expression of POU5-F1 itself in adult stem cells.

### Example 2

### Culturing Human Mesenchymal Stem Cells

Adult human mesenchymal stem cells were cultured by standard methods of in-vitro cell culture. MSCs derived from human cord blood were used for these studies (Vitro Diagnostics, Inc Catalog Number SC00A1-1) that were shown to be human by karyotype and PCR analysis of Actin, Cytochrome B and COX1. These cells were free of common human viral contamination and mycoplasma. The cells exhibited differentiation capacity to form chondrocytes, adipocytes and osteoblasts. These cells were cultured in low serum medium (Vitro Diagnostics, Inc Catalog number SC00B1) optimized for self-renewal of human MSCs. Cells were plated at 10,000 cells per cm² in T12.5 (Flacon, catalog number 353107) previously coated with poly-L-lysine (Sigma catalog number P-1399) at 2 μg/cm². These culture flasks were incubated in an humidified chamber at 37° C. in a gas phase consisting of 20% O₂, 5% CO₂, balance N₂ or a gas phase consisting of 5% O₂, 5% CO₂, balance N₂. Cultures were fed after three days and then sub-cultured at ˜90% confluence using Accutase™ (Innovative Cell Technologies, Inc.) for detachment of the cells. The detached cells were washed out with PBS (5 ml then 3 ml) and the mixture was centrifuged at 450×G for 7 minutes. The cells were aspirated and resuspended in 1 ml PBS. Cells were diluted 1/200 in Isoton-II (Beckman Coulter) and counted in the Z2 Particle Counter (Beckman Coulter). Doubling time for a particular passage was calculated at (ln2*▴T)/(lnC_{f}/Cᵢ) where ▴T is the time from plating of the cells to sub-culture in hours and Cᵢ is the initial number of cells added to the flask and C_{f} is the final number of cells recovered by sub-culture.

### Example 3

### Effects of Reduced Oxygen Environment on Adult Stem Cells

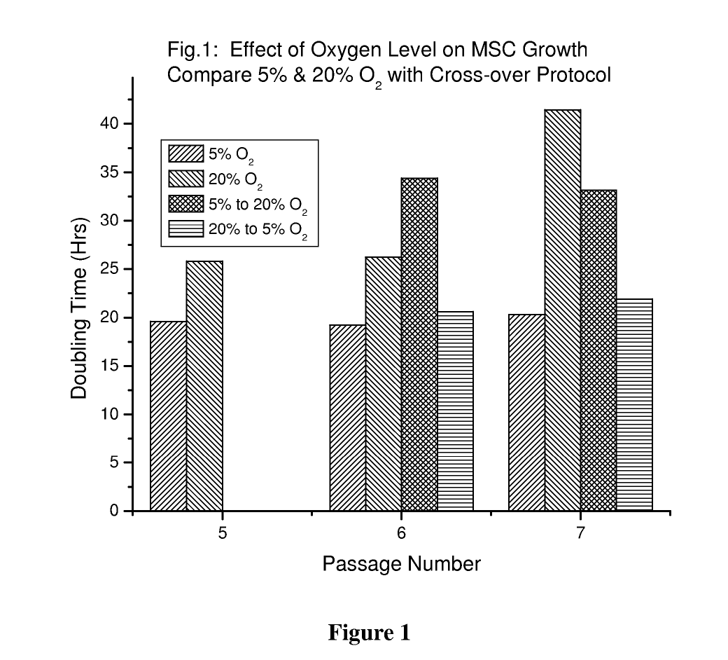

FIG. 1 shows the effects of a reduced oxygen environment on the proliferation of human mesenchymal stem cells. Cells were cultured in either 5% O₂ or 20% O₂ and were also “crossed over” from 5% to 20% O₂ and 20% and 5% O₂ following passage 4. MSCs maintained in 5% O₂ grew rapidly with a doubling time of about 20 hours that was consistently maintained throughout three successive passages. However, MSCs maintained at 20% O₂ grew more slowly in the initial two passages, Td about 25 hours and then slowly considerably in the third successive passage (Td 40 hours). There were 8-fold more cells recovered in passage 7 at 5% O₂ than at 20% O₂. The cross-over results showed reversion to more rapid growth of cells originally exposed to 20% O₂ when these cells were subsequently cultured in 5% O₂. Likewise, cells that were originally cultured in 5% O₂ slowed when exposed to 20% O₂ to rates similar to those seen in cells exposed to 20% O₂ throughout. Thus, these results confirm that the effects seen are due to changes in O₂ content between the two different experimental conditions.

These results disclose that elevated oxygen is toxic to human mesenchymal stem cells and that self renewal is optimally maintained in reduced oxygen environments. Thus, in the remaining examples, MSCs were cultured in a 5% O₂ environment.

### Example 4

### Transfection of Adult Stem Cells with POU5-F1 (Oct3/4)

The inventor determined the effect of over-expression of POU5F1 (Oct3/4) on human mesenchymal stem cells.

Lentiviral Expression Vector Production:

The expression clone containing the human POU5-F1 sequence (Gen Bank # Z11898.1; GeneCopeia™ vector EX-Z0092-Lv105), the CMV promoter and the puromycin stable selection marker was extracted and transduced into GCI-L3 chemically competent E. coli and expanded according to the manufacture's recommended procedures. The Qiagen HiSpeed Plasmid Purification kit (catalog number 12643) was used to purify the plasmid from lysed bacteria. Plasmids were characterized by their UV absorption spectrum. Pseudovirus particles were generated in 293Ta cells that were expanded and transfected using the Lenti-Pac™ HIV Expression Packing Kit (GeneCopoeia Catalog number HPK-LvTR-20). The viral titer was estimated in human MSC cells (Vitro Diagnostics, Inc., catalog number SC00A1) using a control vector expressing GFP and fluorescent analysis.

Transfection of Human MSC & Selection of Transfectants:

Human mesenchymal stem cells (Vitro Diagnostics, Inc., catalog number SC00A1) were cultured at 7500/cm² in T25 flasks (Greiner Bio-One catalog number 690-190) following coating at 2 μg/cm² with poly-L-lysine (Sigma Catalog number P1399) using low serum complete MSC cell culture medium (Vitro Diagnostics, Inc., catalog number SC00B1). Cultures were maintained here and throughout this study in a 5% O₂, 5% CO₂, balance N₂ gas environment in a humidified chamber maintained at 37° C. Following two days of continuous culture and at about 40% confluence, the cultures were exposed to 1/15 dilution of the POU5-F1 pseudovirus stock in low serum medium containing 5 μg/ml polybrene (Sigma catalog number H9268). Following 10 hours exposure to the pseudovirus particles, the cultures were washed twice with PBS and cultured in low serum medium (Vitro SC00B1). Selection of stable transfectants began 16 hours later using 3 to 10 μg/ml puromycin (Sigma catalog number P7255) which was a sufficient concentration to kill native, non-transfected human MSC. Single colony clones were established by limited dilution cloning in 96 well plates (Greiner Bio-One, catalog number 655-160) using low serum MSC medium (Vitro Diagnostics, Inc., catalog number SC00B1) containing 10 μg/ml puromycin.

Initial studies showed that native human MSC were sensitive to puromycin. Dose-response experiments showed that >/=3 μg/ml puromycin in the medium effectively killed native MSC. Since the inventor routinely used 10 μg/ml puromycin for expansion of clones, it is highly likely that only those cells expressing puromycin resistance grew in this medium.

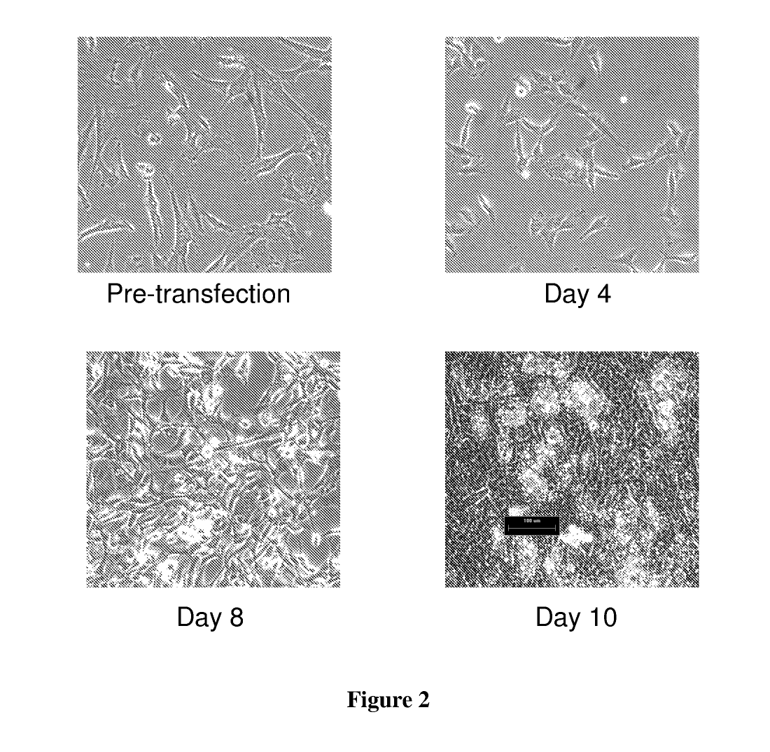

Limited dilution cloning was used to isolate and expand single colonies. The inventor was able to isolate and expand about 15 individual clones using this method. FIG. 2 shows the selection of transfectants by lethal dosage of puromycin. Pre-transfection shows classic MSC morphology and this was maintained until about day 10 when differentiated structures similar in morphology to adipocytes were apparent. These cultures were typically composed primarily of mesenchymal cells. However, there were also apparently differentiated structures, especially resembling the chondrocytic lineage. These structures were dependent on the environment used for expansion and this characteristic was also similar to non-transfected MSCs.

### Example 5

### Characterization of Adult Stem Cell Transfectants

Self-renewal was determined by expansion of isolated single colonies that resulted from transfection and selection of transfectants as described above. These were initially established in 96-well plates and then sub-cultured using Accutase (Innovative Cell Technologies, Inc catalog number AT104) followed by washout with PBS and transfer to single wells of 48-well plates (Greiner Bio-One catalog number 677 180). The following sub-culture was into T12.5 flasks (Beckton Dickinson catalog number 353107). All cultures were maintained in poly-L-lysine coated cultureware (described above) using low serum medium (Vitro Diagnostics, Inc., catalog number SC00B1) containing 10 μg/ml puromycin. Growth rates were determined by cell counting and calculation of doubling time.

Differentiation capacity into the chondrogenic, adipogenic and osteogenic lineages was determined by parallel culture of native MSC and transfectants within 48 well plates (Greiner Bio-One catalog number 677 180). Chondrogenesis occurred without medium additions while adipogenesis was induced by a medium comprising low serum MSC medium (Vitro Diagnostics, Inc., catalog number SC00B1) containing 1 μM dexamethasone (Sigma catalog number D2915), 0.5 mM 3-isobutyl-1-methylxanthine (Sigma catalog number 1708) and 0.2 mM indomethacin (Sigma catalog number 18280). Osteogenesis was induced by a medium comprising low serum MSC medium (Vitro Diagnostics, Inc., catalog number SC00B1) containing 3 μM purmorphamine (StemGent catalog number 04-0009). Cultures were maintained in continuous culture for 12 to 18 days with feeding every 2-3 days at which time the cultures were fixed in 4% buffered formaldehyde for 30 minutes at room temperature and subsequently washed three times with PBS. Proteoglycans were determined by Alcian blue staining (1% in 0.1 N HCl) for 30 minutes, lipids were detected by Oil Red staining (0.5% in isopropyl alcohol) for one hour and osteoblasts were detected by Alizarin Red S staining of mineralized matrix (1% in 2% EtOH) for five minutes according to Kulterer, et al, 2007.

Stem cell potency assay was performed using the Lumi-STEM™-96 assay (Hemogenix, Inc. catalog number KLS-96-A-2). Cell samples were prepared from expanding cultures by sub-culture at the dilutions indicated (see results) these were then added to white 96 well plates and incubated in 5% O₂, 5% CO₂, in an humidified chamber maintained at 37° C. for 1 hour prior to addition of ATP reagent.

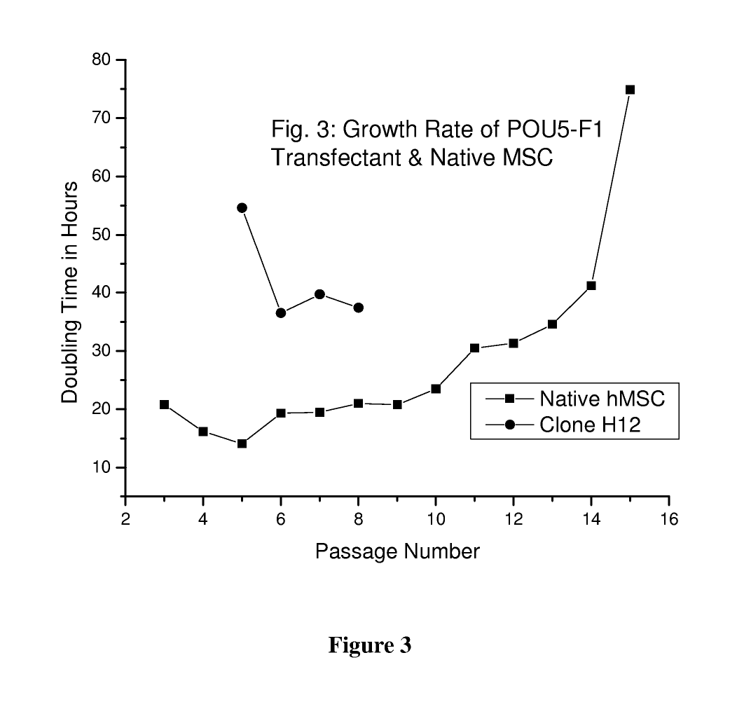

FIG. 3 shows growth analysis of clone H12 (transfected with POU5-F1) and native MSC. Growth of the H12 clone was slow initially and then increased but remained slower than native MSC throughout the period of expansion examined in this study. Also, the transfectants appeared to be larger than native MSC.

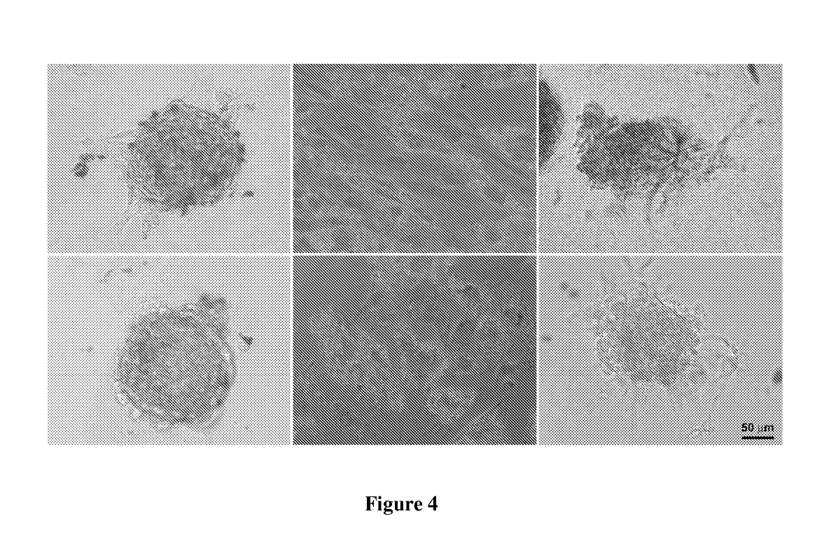

FIG. 4 shows results of the comparison of chondrogenic, adipogenic and osteogenic differentiation capacity of a transfected cell line (lower panels, Clone A7) and native MSC (upper panels) according to the Methods previously described. Staining with Alcian Blue (left panels) and Oil Red O (middle panels) showed similar results for both transfectants and native MSC, suggested comparable multipotent differentiation capacity. Results of osteogenic differentiation (right panels) also suggest comparable differentiation although there only a phase contrast image of native MSC showing possible mineral matrix structures.

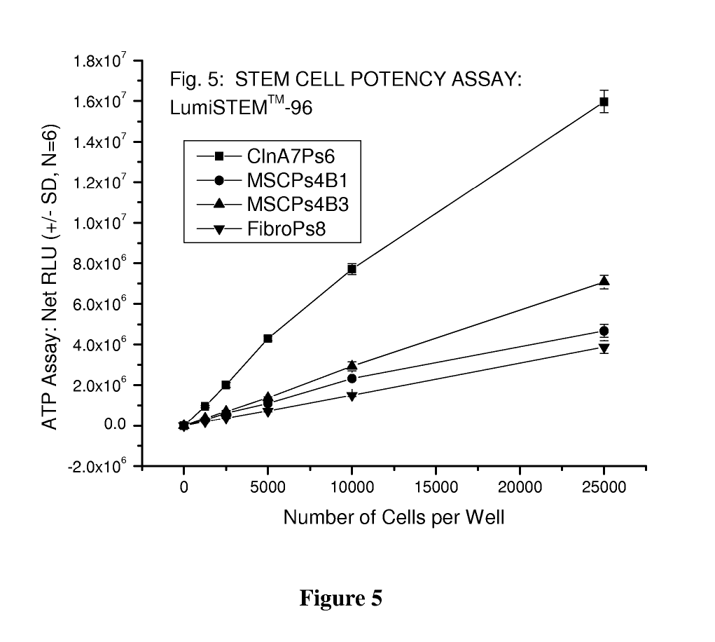

Previous results suggested possible expanded differentiation capacity of transfectants as compared to native MSC since adipocyte-like cells appeared following transfection without exposure to differentiating agents (e.g., PPAR-γ agonists) as shown in FIG. 2. The inventor thus determined stem cell potency by a quantitative method based on ATP determinations and the results are shown in FIG. 5.

Results of the LumiSTEM™ assay showed a greater slope of the cellular dose-response curve for clone A7 than native MSC grown in serum-free medium (Vitro Biopharma catalog number SC00B3), p<0.0001 by analysis of Covariance (ANCOVA, Graphpad, Prism™). Native MSC also showed a greater slope than a primary human fibroblast cell line. These results also support the hypothesis that POU5-F1 transfected human MSC exhibit greater differentiation capacity than native MSC.

### Example 6

### Small Molecular Agents Optimize Pluripotency of Adult Stem Cells

The following small molecules when added to the medium are expected to result in increased POU5F1 (Oct3/4) expression: 1) Hydrocortisone preferable at 80 to 110 nM, 2) valproic acid at 1 to 2 mM, 3) Vitamin C at 60 μM, 4) 5-azacytidine at 0.5 mM , especially for transient periods </=48 hours, 5) sodium butyrate at 1 to 2 mM, and 6) GnRH at 75 pg/ml. Also, it is also apparent that microRNA complexes positively regulate the expression of Oct4, in particular the miR-290-295 cluster. Hence, expression of Oct4 in adult stem cells through introduction of the microRNA family known as the miR-290-295 cluster is possible.

### Example 7

### One Embodiment of the Present Invention

One embodiment of the present invention comprises use of human or animal MSCs derived from bone marrow, cord blood, Warton's jelly, adipose tissue, teeth, amniotic fluid or placental tissues and maintained within a cell culture medium capable of maintaining doubling times of 20 to 30 hours through 5 successive passages. The cell culture environment includes a gas phase of 1% O₂, appropriate CO₂ levels to achieve physiological pH in the cell culture medium with the balance of the gas phase comprised of N₂. The cell culture medium also contains 10⁻⁷ M hydrocortisone.

As further needed to induce pluripotency, cell culture medium also contains 1.0 mM to 2.0 mM valproic acid for a period of 8 to 10 days of culture which is thereafter removed from the medium. In the event that the pre-iPS condition does not progress to complete iPS programming or this occurs only at low frequency, then 0.5 mM 5-azacytidine is added to the culture medium for 48 hours to facilitate higher efficiency of pluripotent reprogramming Said pluripotent cells produced by this method are used for various applications in drug discovery and development and therapeutic applications. It will be obvious to those skilled in the art that specific applications may require terminally differentiated cells that may be conveniently derived from said pluripotent stem cells through use of various conditions necessary to induce differentiation into specific cell lineages. Therapeutic applications include, but are not limited to, treatment of joint diseases or injury, heart attack, stroke, macular degeneration and age-related hearing loss.

The principle and mode of operation of this invention have been explained and illustrated in its preferred embodiment. However, it must be understood that this invention may be practiced otherwise than as specifically explained and illustrated without departing from its spirit or scope.

What is claimed is:

1. A method to culture cells, comprising culturing adult stem cells at less than 20% v/v oxygen so as to induce pluripotency.

2. A method of claim 1, wherein the adult stem cells are cultured at from about 1% v/v to about 5% v/v oxygen.

3. A method of claim 2, wherein the adult stem cells are cultured at from about 1% v/v to about 3% v/v oxygen.

4. A method of claim 3, wherein the adult stem cells are cultured at from about 1% v/v to about 2% v/v oxygen.

5. A method of claim 4, wherein the adult stem cells are cultured at approximately 1% v/v oxygen.

6. A method of claim 1, wherein the balance of the gas phase is nitrogen.

7. A method of claim 1, which further comprises culturing in the presence of a small molecule that promotes expression of POU5-F1 (Oct3/4).

8. A method of claim 7, wherein the small molecule is selected from the group consisting of: valproic acid; 5-azacytidine; sodium butyrate; ascorbic acid (Vitamin C); gonadotropin releasing hormone; antisense or sense miRs of the miR-290-295 cluster; and hydrocortisone.

9. A method of claim 1, wherein the adult stem cells are selected from the group consisting of: hematopoietic stem cells; mesenchymal stem cells of any origin including: bone marrow, adipose, umbilical cord, peripheral blood, Warton's jelly of the umbilicus, skin, decidua of the placenta, amniotic fluid, teeth (both juvenile and adult); neural stem cells, olfactory epithelium, and peripheral neural stem cells; muscle satellite cells; endocrine-specific adult stem cells; and endothelial adult stem cells.

10. A method of claim 5, wherein the adult stem cell is a mesenchymal stem cell.

11. A method of claim 1, which further comprises a step of identifying pluripotency attributes of the cultured cells.

12. A method of claim 1, which further comprises a step of purifying pluripotent cells.

13. A method of claim 1, which further comprises a step of identifying POU5-F1 (Oct3/4) expression.

14. A product made by the process of claim 1.

15. A method to identify useful therapeutic compounds, comprising culturing a cell according to the method of claim 1, introducing a test therapeutic compound, and determining if the test therapeutic compound is useful.

16. A method to treat a disease or injury, comprising administering a product of claim 14.

17. A method of claim 16, wherein the disease or injury is selected from the group consisting of: leukemia; lymphoma; diabetes; Alzheimer's; Parkinson's; multiple sclerosis; osteoarthritis; stroke; myocardial infarction; congestive heart failure; graft-verses-host disease; traumatic brain injury; Crohn's disease; stem cell-mediated malignancy; age-related hearing loss; macular degeneration; spinal cord injury; end-stage renal disease; acute or chronic renal failure; diabetes-related cardiovascular disease; ALS; spinal cord injury; herniated disc; ligament or tendon rupture.

18. A method to make a differentiated cell, comprising differentiating a product of claim 14.

19. A method of claim 18, wherein the differentiated cell is selected from the group consisting of: cardiomyoctes; neuronal cells; neurotransmitter-specific neurons; sensory neurons; alpha motor neurons; Schwann and glial cells; hepatocytes; vascular endothelial cells; skeletal cells; smooth muscle cells; renal tubule cells; glomerular cells; kerotinocytes; osteoblasts; adipocytes; chondrocytes; pituitary hormone producing cells; thyroid cells; adrenal cells; melanocytes; thymocytes; erythrocytes; neutrophils; basophils; eosinophils; macrophages, platelets; T-lymphocytes; B-lymphocytes; gut epithelial cells; urogenital tract epithelial cells.

20. A product made by the process of claim 19.
